(19) United States
(12) Patent Application Publication
(10) Pub. No.: US 2009/0253957 A1
(43) Pub. Date: Oct. 8, 2009

(54) ENDOSCOPE APPARATUS

(75) Inventor: Shinji YASUNAGA, Asaka-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/416,362

(22) Filed: Apr. 1, 2009

(30) Foreign Application Priority Data
Apr. 4, 2008 (JP) JP 2008-098521

(51) Int. Cl. A61B 1/04

(52) U.S. Cl. 600/109

(57) ABSTRACT

An endoscope apparatus of the present invention includes a scope portion having a rigid portion in which, at least an image pickup module is disposed, a circulation passage which is at an interior of the scope portion, and which is extended rearward from the rigid portion, such that a cooling medium filled inside is capable of carrying out a heat exchange with the rigid portion, and a pump which is at the interior of the scope portion, and which circulates the cooling medium inside the circulation passage.

### CROSS-REFERENCE TO RELATED APPLICATION

The present application is based upon and claims the benefit of priority from the prior Japanese Patent Application No. 2008-98521 filed on Apr. 4, 2008; the entire contents of which are incorporated herein by reference.

### BACKGROUND OF THE INVENTION

1. Field of the Invention

The present invention relates to an endoscope apparatus.

2. Description of the Related Art

With a progress in a semiconductor technology, there has been an improvement in the number of pixels and a frame rate of an image pickup element of an endoscope apparatus, and an observation of a diseased part by a high-quality image has been realized. Meanwhile, the image pickup element causes an increase in heat generation due to a high performance, and an improvement in the image quality is hindered due to an increase in a thermal noise. For this reason, a realization of an image pickup element cooling mechanism which can be mounted on an image pickup module of an endoscope apparatus has been sought.

As an endoscope which includes such image pickup element cooling mechanism, an endoscope disclosed in Japanese Patent Application Laid-open Publication No. 2006-664 has hitherto been known. This endoscope will be described below by using FIG. 12. FIG. 12 is a cross-sectional view showing a structure of a conventional endoscope. In a scope portion 200 of the endoscope shown in FIG. 12, two pipes 220 for cooling medium reflux (hereinafter, ‘cooling medium reflux pipes 220’) inserted into an inserting section and wound round an outer peripheral surface of an image pickup unit 211 form a cooling section 221. The image pickup unit 211 is cooled by sending a cooling medium from one of the two cooling medium reflux pipes 220 and refluxing (returning) the cooling medium in a ring form by the cooling section 221, and is recovered in hands of an endoscope operator through the other cooling medium reflux pipe 220.

Moreover, a thermistor 222 of which, a resistance changes with a temperature for example, is provided as a temperature detecting sensor which detects a temperature of the image pickup element is provided at an interior of the image pickup unit 211. By measuring the resistance of the thermistor 222, a judgment of whether or not the temperature has become a predetermined temperature or higher than the predetermined temperature, is made. When the temperature has become the predetermined temperature or higher than the predetermined temperature, an operation of refluxing the cooling medium through the cooling medium reflux pipe 220 is carried out, and the image pickup unit 211 is cooled.

In other words, a cooling means wound around the cooling medium reflux pipe 220 is provided to the image pickup unit 211, and the temperature detecting means is provided at the interior of the image pickup element 211. With such an arrangement, by carrying out the cooling operation by the cooling means when the predetermined temperature or the temperature higher than the predetermined temperature is attained, the heat generation of a rigid portion 210 is prevented.

However, in the endoscope disclosed in the Japanese Patent Application Laid-open Publication No. 2006-664, for recovering the cooling medium at the hands of the endoscope operator, a pump which sends the cooling medium to an outside of the scope portion 200 is necessary. It is possible to use this pump also as a water supply system for cleaning a diseased part and a lens surface. However, in such a case, a valve mechanism is necessary inside or outside the scope portion 200, and a mechanism such as a switching mechanism may become complicated. Furthermore, in an endoscope such as an endoscope for a bronchial tube, which does not necessitate a water supply mechanism other than for cooling the image pickup element, a structure as an endoscope system may become complicated.

### SUMMARY OF THE INVENTION

The present invention is made in view of the abovementioned circumstances, and an object of the present invention is to provide an endoscope apparatus which includes a cooling mechanism having a simple structure and a favorable efficiency.

To solve the abovementioned issues and to achieve the object, the endoscope apparatus according to the present invention includes

a scope portion having a rigid portion in which, at least an image pickup module is disposed,

a circulation passage which is at an interior of the scope portion, and which is extended rearward from the rigid portion, such that a cooling medium filled inside is capable of carrying out a heat exchange with the rigid portion, and

a pump which is at the interior of the scope portion, and which circulates the cooling medium in the circulation passage.

In the endoscope apparatus according to the present invention, it is preferable that the scope portion includes a bending portion which is positioned rearward of the rigid portion, and which is bendable by an operation by an operator, and a flexible portion which is positioned rearward of the bending portion, and at least a part of the circulation passage is flexible, and the circulation passage is extended at least up to the bending portion.

In the endoscope apparatus according to the present invention, the rigid portion may have a mounting substrate on which, at least an image pickup element is mounted, and a part of the circulation passage may be joined to the mounting substrate.

In the endoscope apparatus according to the present invention, the pump may be disposed inside the rigid portion.

In the endoscope apparatus according to the present invention, it is preferable that the pump is joined to the mounting substrate.

In the endoscope apparatus according to the present invention, the circulation passages may be extended up to the flexible portion, and the pump may be disposed in the flexible portion.

In the endoscope apparatus according to the present invention, it is desirable that a thermoelectric cooling element is disposed inside the rigid portion, and a part of the circulation passage is joined to a heat releasing surface of the thermoelectric cooling element.

In the endoscope apparatus according to the present invention, the cooling medium may be an electro-conjugate fluid, and the pump may send the electro-conjugate fluid by applying an electric field to the electro-conjugate fluid.

An endoscope according to the present invention includes

a scope portion having a rigid portion in which, at least an image pickup module is disposed,

a heat absorbing portion which is capable of carrying out heat exchange with the rigid portion,

a circulation passage which is extended rearward from the heat absorbing portion, at an interior of the scope portion, and in which a cooling medium is filled, and

a pump which is at the interior of the scope portion, and which circulates the cooling medium in the circulation passage.

### BRIEF DESCRIPTION OF THE DRAWINGS

FIG. 1 is a perspective view showing an internal structure of a scope portion of an endoscope apparatus according to a first embodiment;

FIG. 2 is a perspective view showing a structure of a cooling unit according to the first embodiment;

FIG. 3 is a plan view showing a structure of the cooling unit according to the first embodiment;

FIG. 4A is a front view showing a rigid portion of the scope portion, and FIG. 4B is a cross-sectional view taken along a line IVB-IVB in FIG. 4A;

FIG. 5 is a perspective view of an internal structure of a scope portion of an endoscope apparatus according to a second embodiment of the present invention;

FIG. 6 is a perspective view showing a structure of a cooling unit according to the second embodiment of the present invention;

FIG. 7 is a perspective view showing a structure of a cooling medium circulating pump according to the second embodiment;

FIG. 8 is a plan view showing a channel substrate according to the second embodiment;

FIG. 9A is a front view showing a structure of a rigid portion of a scope portion, and FIG. 9B is a cross-sectional view taken along a line IXB-IXB in FIG. 9A;

FIG. 10 is a perspective view showing an internal structure of a scope portion of an endoscope apparatus according to a third embodiment of the present invention;

FIG. 11 is a perspective view showing an internal structure of a scope portion of an endoscope apparatus according to a fourth embodiment; and

FIG. 12 is a cross-sectional view showing a structure of a conventional endoscope

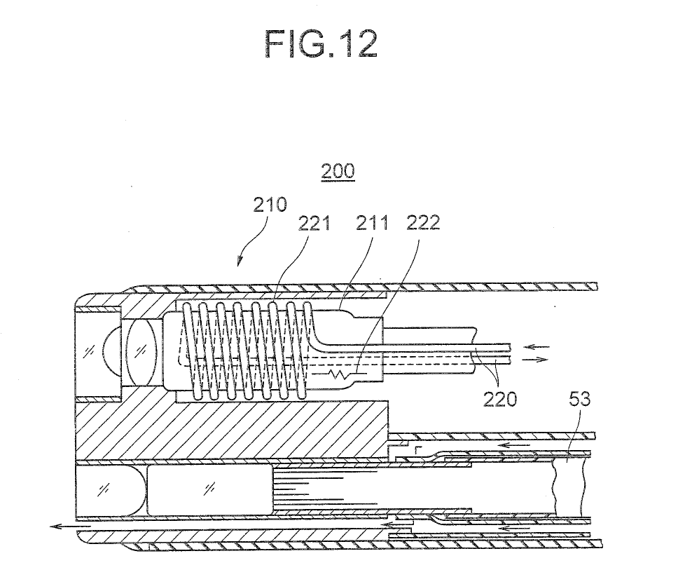

### DETAILED DESCRIPTION OF THE PREFERRED EMBODIMENTS

Exemplary embodiments of an endoscope apparatus according to the present invention will be described below by referring to the accompanying diagram. However, the present invention is not restricted to the following embodiments

### First Embodiment

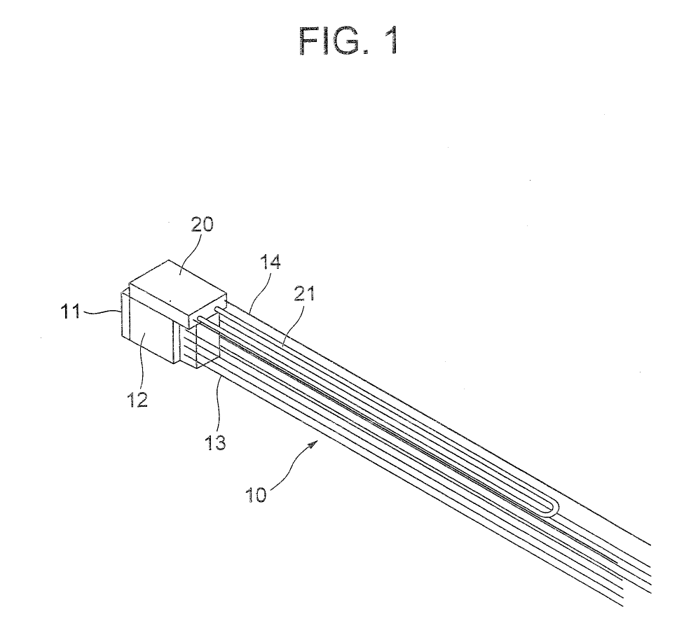

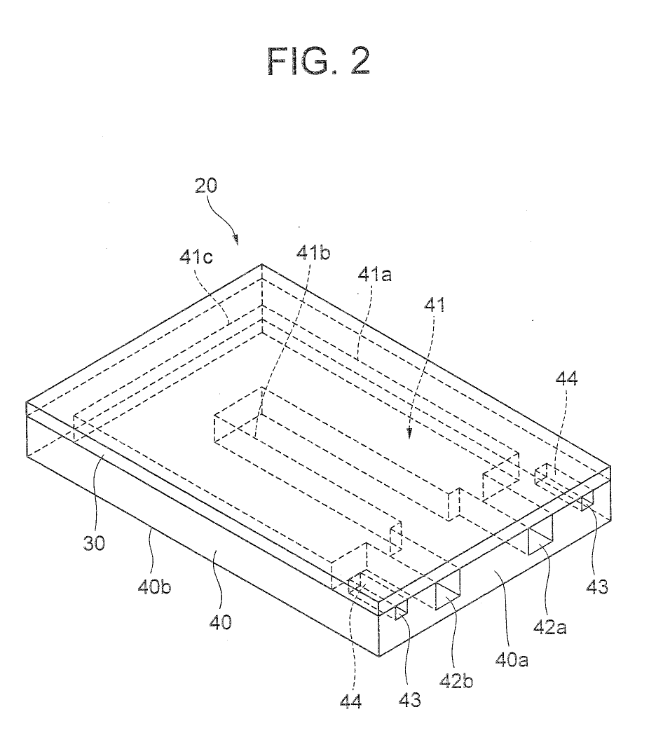

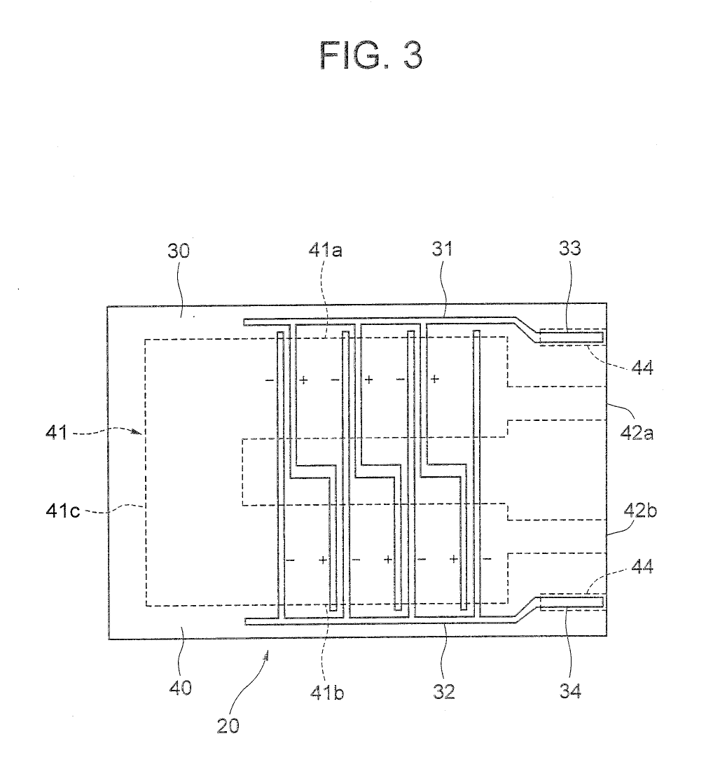

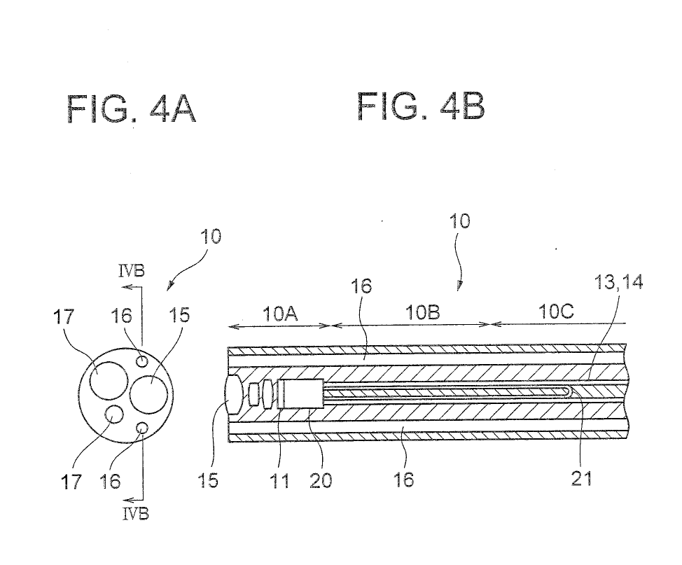

An endoscope apparatus according to a first embodiment of the present invention will be described below by referring to diagrams from FIG. 1 to FIG. 4B. FIG. 1 is a perspective view showing an internal structure of a scope portion 10 of the endoscope apparatus according to the first embodiment, and is a diagram showing a structure of an image pickup module including an image pickup element 11. FIG. 2 is a perspective view showing a structure of a cooling unit 20 (heat absorbing portion and pump) according to the first embodiment. FIG. 3 is a plan view showing a structure of the cooling unit 20, and is a diagram when seen from a top a driving substrate 30. FIG. 4A is a front view showing a structure of a rigid portion 10A of the scope portion 10, and FIG. 4B is a cross-sectional view taken along a line IVB-IVB in FIG. 4A, and is a diagram showing the internal structure of the scope portion 10. In the following description, regarding a structure other than the scope portion 10 in the endoscope apparatus, hitherto known structure can be used, and therefore a description thereof in detail is omitted.

The image pickup element 11 is mounted on a mounting substrate 12, and a cooling unit 20 is stuck to one of side surfaces of the mounting substrate 12. Moreover, it is not shown particularly in the diagram but, a number of components forming a peripheral circuit of components such as a driver chip of the image pickup element 11 are mounted on the mounting substrate 12. Further, a plurality of lead wires 13 is connected to a rear-end portion of the mounting substrate 12, and a wire for driving (hereinafter, ‘driving wire’) 14 and a cooling-medium circulation tube 21 (circulation passage) are extended rearward from the cooling unit 20. In the following description, a front side of the scope portion 10 refers to a side on which a lens 15 is disposed, in a direction in which the scope portion 10 is extended (left side in FIG. 4B), and a rear side of the scope portion 10 refers to an operator side which is away from the lens 15 (right side in FIG. 4B). An image pickup module of the first embodiment includes the image pickup element 11 and the mounting substrate 12.

Next the structure of the cooling unit 20 will be described below while referring to FIG. 2. In FIG. 2, an anode electrode 31 and a cathode electrode 32 which will be described later are omitted. The cooling unit 20 has a form in which, a driving substrate 30 and a channel substrate 40 are stuck. A channel 41 (circulation passage) is formed as a recess in the channel substrate 40. The channel 41 has a structure in which, substantially symmetric channels namely a first channel 41a and a second channel 41b communicate, sandwiching a turning channel 41c. Two ends of the channel 41, in other words, end portions of the first channel 41a and the second channel 41b are two tube inserting holes 42a and 42b communicating with an outside, at one end surface 40a of the channel substrate 40.

Moreover, two raceways 44 are formed in the channel substrate 40 as recesses differing from the channel 41. One end of each raceway 44 is a wire inserting hole 43 communicating with the outside, in the end surface 40a in which, the tube inserting holes 42a and 42b are formed.

Both ends of the cooling-medium circulation tube 21 are inserted into the tube inserting holes 42a and 42b. Moreover, the driving wire 14 is inserted into each wire inserting hole 43. In the cooling unit 20 having the abovementioned structure, a bottom surface 40b of the channel substrate 40 is stuck to the mounting substrate 12. Furthermore, an electro-conjugate fluid is filled in the channel 41 and the cooling-medium circulation tube 21.

Next, a structure of the driving substrate 30 will be described below by referring to FIG. 3. The driving substrate 30 includes the anode electrode 31 and the cathode electrode 32 made of a metal formed on a glass substrate. The anode electrode 31 is extended up to a portion above an anode-side wire connecting portion 33, and the cathode electrode 32 is extended up to a portion above a cathode-side wire connecting portion 34. Further, the anode-side wire connecting portion 33 and the cathode-side wire connecting portion 34 are connected to the driving wires 14 to be inserted into the two raceways 44 respectively.

As shown in FIG. 3, the anode electrode 31 and the cathode electrode 32 form interdigital electrodes which cross mutually on the first channel 41a and the second channel 41b. When a high voltage is applied between the anode-side wire connecting portion 33 and the cathode-side wire connecting portion 34, a high electric field pointing left is applied on the first channel 41a and a high electric field pointing right is applied on the second channel 41b. The channel 41 being turned by the turning channel 41c, a high electric field in the same direction is applied discretely along the channel, in other words, along the channel directed from the tube inserting hole 41a to the tube inserting hole 42b. Therefore, when a high voltage is applied between the two driving wires 14, a flow of the electro-conjugate fluid is generated by the high electric field in the channel 41, and the electro-conjugate fluid is circulated by the cooling-medium circulation tube 21.

In this manner, the cooling unit 20 is a pump which circulates the electro-conjugate fluid in the channel 41 and the cooling-medium circulation tube 21. Here, it is preferable to form the channel substrate 40 to be stuck to the mounting substrate 12 of a material having a high coefficient of thermal conductivity (such as alumina) and to form the driving substrate 30 of a material having a low coefficient of thermal conductivity (such as glass). Moreover, it is possible to apply a photolithography technology used in semiconductor manufacturing, for forming the anode electrode 31 and the cathode electrode 32. Accordingly, it is possible to make small the cooling unit 20. Thus it is possible to dispose the cooling unit 20 in the rigid portion 10A having a substantial constraint of space due to a large number of members existing therein.

Next, a structure when the scope portion 10 is disposed in a concrete endoscope apparatus will be described below while referring to FIG. 4.

The scope portion 10 includes the rigid portion 10A of which, a front portion does not bend, a bending portion 10B of which, an outer shell is formed of a bending piece, and which is bendable by wire traction, and a flexible portion 10C which is flexible. Taking into consideration an operability inside a body cavity, it is desirable that the rigid portion 10A which does not bend is as short as possible. The image pickup element 11, the lens 15 disposed in front of the image pickup element 11, a light guide 16, a channel 17 through which forceps etc. are inserted, the cooling unit 20, and the cooling-medium circulation tube 21 are disposed in the scope portion 10.

The cooling unit 20, by circulating the electro-conjugate fluid, suppresses a rise in temperature inside the rigid portion 10A due to heat generation from components in a surrounding circuit (not shown in the diagram) mounted on the mounting substrate 12 and the image pickup element 11. Further, by circulating the electro-conjugate fluid in the cooling-medium circulation tube 21 extended rearward from the rigid portion 10A, it is possible to release the heat exchanged at the rigid portion 10A, to the surrounding via the cooling-medium circulation tube 21. Accordingly, since it is possible to achieve a heat releasing effect at a rearward of the rigid portion 10A in addition to an effect of suppressing the rise in temperature by the cooling unit 20 inside the rigid portion 10A having a limited volume, it is possible to achieve a substantial cooling effect. For achieving a high heat-release effect, it is preferable that the cooling-medium circulation tube 21 is as long as possible. A length of the cooling-medium circulation tube 21 can be set in accordance with an amount of heat generated by the image pickup element 11, a flow velocity of the electro-conjugate fluid, and a material and a diameter of the cooling-medium circulation tube 21, and when the length of the cooling-medium circulation tube 21 is let to be in a range of 10 cm to 50 cm, it is possible to achieve a substantial heat-release effect. In the first embodiment, since the cooling-medium circulation tube 21 is flexible and is extended inside the bending portion 10B, it is possible to suppress to the minimum an increase in a size in a radial direction of the scope portion 10 and to incorporate a cooling mechanism having a favorable efficiency.

### Second Embodiment

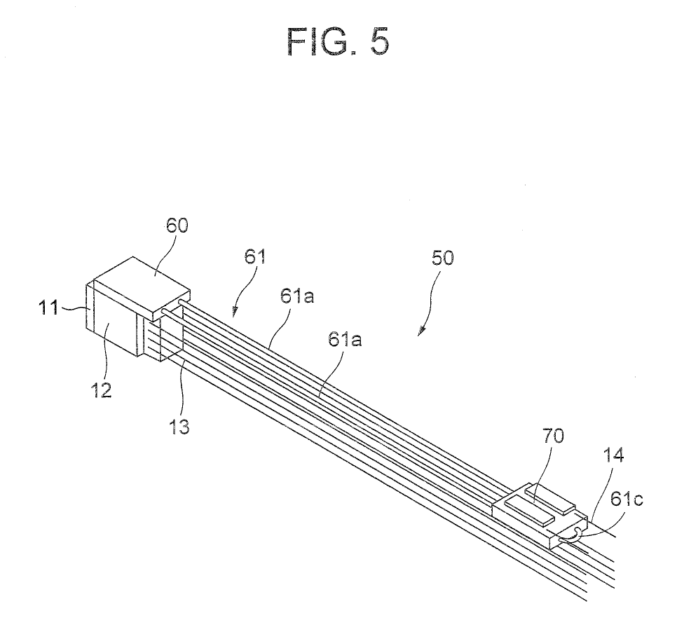

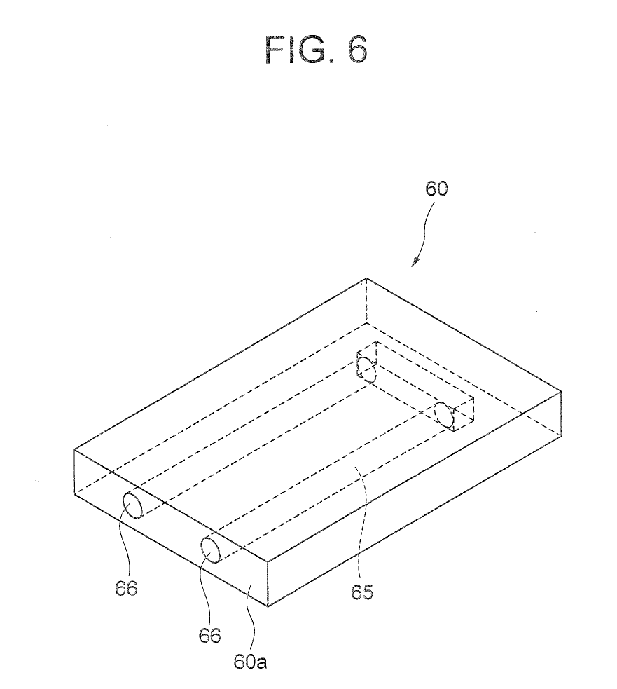

Next, an endoscope apparatus according to a second embodiment of the present invention will be described below by referring to diagrams from FIG. 5 to FIG. 9. FIG. 5 is a perspective view showing an internal structure of a scope portion 50 of the endoscope apparatus according to the second embodiment, and is a diagram showing a structure of an image pickup module including the image pickup element 11. FIG. 6 is a perspective view showing a structure of a cooling unit 60 (heat absorbing section) according to the second embodiment.

In the endoscope apparatus according to the second embodiment, a point that, a cooling-medium circulating pump 70 separate from the cooling unit 60 is disposed in a flexible portion 50C of the scope portion 50 differs from the endoscope apparatus according to the first embodiment. In the following description, same reference numerals are assigned to members that are same as in the first embodiment.

In the scope portion 50, the cooling unit 60 is stuck for cooling the mounting substrate 12, and cooling-medium circulation tube 61 (circulation passage) is extended rearward from the cooling unit 60. The cooling-medium circulating pump 70 is disposed half-way at a rearward side of the cooling-medium circulation tube 61, and the pump driving wire 14 is drawn from the cooling-medium circulating pump 70. Moreover, water is sealed as a cooling medium in the cooling unit 60, the cooling-medium circulating pump 70, and the cooling-medium circulation tube 61. It is also possible to use a fluid other than water as a cooling medium.

A structure of the cooling unit 60 will be described below by referring to FIG. 6. The cooling unit 60 is a member in the form of a block made of a member having a high coefficient of thermal conductivity such as copper, and a cooling-medium circulation channel 65 (circulation passage) is formed at an interior thereof. Two ends of the cooling-medium circulation channel 65 are two tube inserting holes 66 communicating with the outside, in one end surface 60a of the cooling unit 60. Two ends of the cooling-medium circulation tube 61 are connected to the two tube inserting holes 66 respectively. The cooling-medium circulation tube 61 includes a first tube 61a, a second tube 61b, and a third tube 61c. The cooling unit 60 being such a simple structure, it is possible to make a size smaller than a size of the cooling unit 20 of the first embodiment, and it is possible to make the image pickup module even smaller.

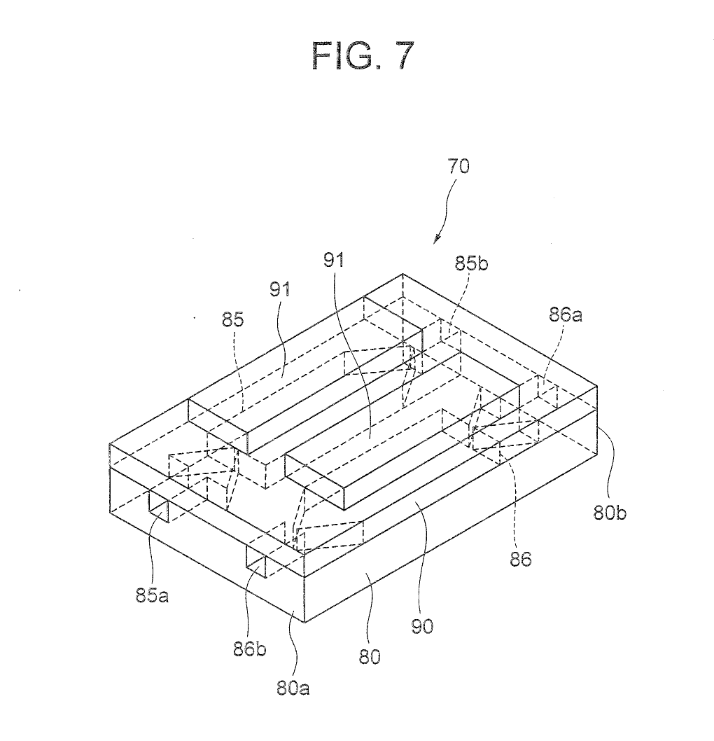

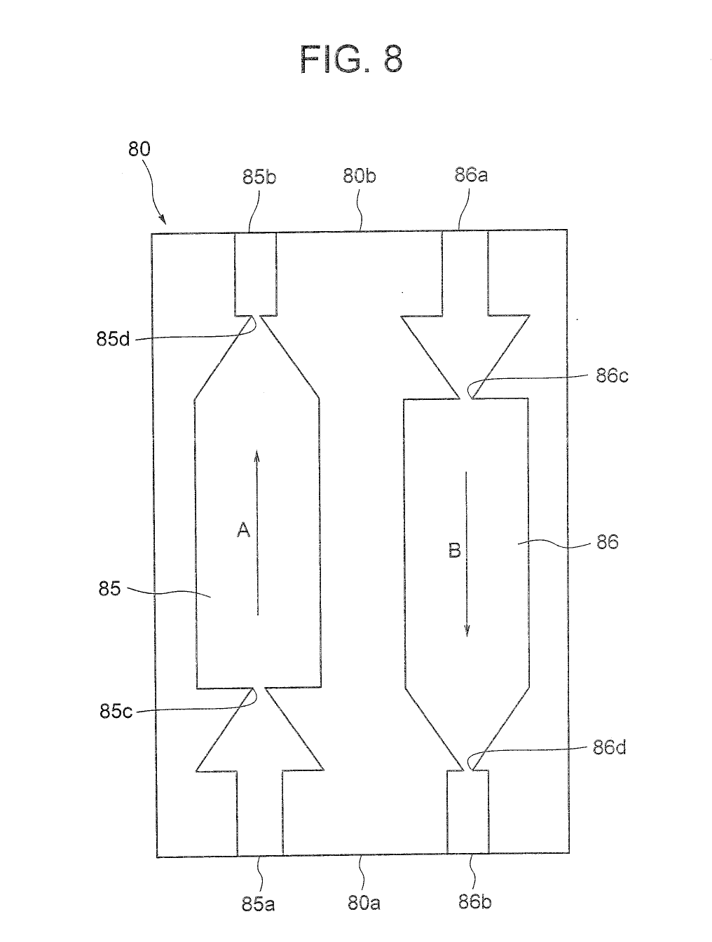

Next, a structure of the cooling-medium circulating pump 70 (pump) will be described below by referring to FIG. 7 and FIG. 8. FIG. 7 is a perspective view showing the structure of the cooling-medium circulating pump 70. FIG. 8 is a plan view showing a structure of a channel substrate 80. The cooling-medium circulating pump 70 includes the channel substrate 80, a vibration substrate 90 stacked on the channel substrate 80, and two piezoelectric vibrators 91 stacked on the vibration substrate 90. Chambers 85 and 86 (circulation passages) are formed in the channel substrate 80 as recesses extended in a longitudinal direction of the channel substrate 80. One end portion of the chamber 85 is an opening 85a communicating with the outside, in an end surface 80a of the channel substrate 80, and the other end portion thereof is an opening 85b communicating with the outside, in an end surface 80b facing the end surface 80b. Similarly, one end portion of the chamber 86 is an opening 86b communicating with the outside, in the end surface 80a of the channel substrate 80, and the other end portion thereof is an opening 86a communicating with the outside, in the end surface 80b.

The cooling-medium circulation tube 61 is connected to the openings 85a, 85b, 86a, and 86b. Concretely, the first tube 61a connected to one tube inserting hole 66 is connected to the opening 85a, and the second tube 61b connected to the other tube inserting hole 66 is connected to the opening 86b. Furthermore, the opening 85b and the opening 86a are mutually connected by the third tube 61c. Consequently, the circulation passage through which the cooling medium is circulated is formed by the cooling-medium circulation channel 65, the cooling-medium circulation tube 61, and the chambers 85 and 86 of the channel substrate 80 of the cooling unit 60.

The cooling-medium circulating pump 70 makes the vibration substrate 90 vibrate in a resonant state by making the piezoelectric vibration 91 thickness-vibrate, and generates a flow of the cooling medium in the chambers 85 and 86 formed in the channel substrate 80. It is not particularly indicated in FIG. 7 but, the pump driving wire 14 connected to the cooling-medium circulating pump 70 is connected to an electrode of the piezoelectric vibrator 91, and makes the piezoelectric vibrator 91 vibrate by a voltage which is applied from outside.

An operation of the cooling-medium circulating pump 70 will be described below while referring to FIG. 7 and FIG. 8. Arrows A and B in FIG. 8 indicate a flow of the cooling medium and not a shape of the channel. As shown in FIG. 8, the channel of the cooling medium is made of the chamber 85 connected to the opening 85a and the opening 85b, and the chamber 86 connected to the opening 86a and the opening 86b. Narrowed portions 85c and 85d are provided between the chamber 85 and the opening 85a, and between the chamber 85 and the opening 85b respectively.

When the piezoelectric vibrator 91 is made to vibrate on the chamber 85 having the abovementioned structure, the vibration substrate 90 also vibrates, and a volume of the chamber 85 changes periodically according to the vibration of the piezoelectric vibrator 91. An amount of the cooling medium discharged during a process of decrease in the volume of the chamber 85 becomes more toward the opening 85b than toward the opening 85a, due to a difference in a nozzle shape. On the other hand, an amount of the cooling medium sucked in during a process of increase in the volume of the chamber 85 becomes more toward the opening 85a than toward the opening 85b. Consequently, in the chamber 85, the flow of the cooling medium is generated in a direction of arrow A.

Whereas, in the chamber 86, narrowed portions 86c and 86d are provided between the chamber 86 and the opening 86a, and between the chamber 86 and the opening 86b. When the vibration substrate 90 is made to vibrate by making the piezoelectric vibrator 91 vibrate on the chamber 86, a volume of the chamber 86 changes periodically according to the vibration of the piezoelectric vibrator 91. An amount of the cooling medium discharged during a process of decrease in the volume of the chamber 86 becomes more toward the opening 86b than toward the opening 86a, due to a difference in a nozzle shape. On the other hand, an amount of the cooling medium sucked in during a process of increase in the volume of the chamber 86 becomes more toward the opening 86a than toward the opening 86b. Consequently, in the chamber 86, the flow of the cooling medium is generated in a direction of arrow B.

The flow of the cooling medium in the direction of arrow A being generated in the chamber 85 and the flow of the cooling medium in the direction of arrow B being generated in the chamber 86 as described above, a circulation is generated in the cooling medium inside the cooling-medium circulation tube 61 and the cooling unit 60 shown in FIG. 5.

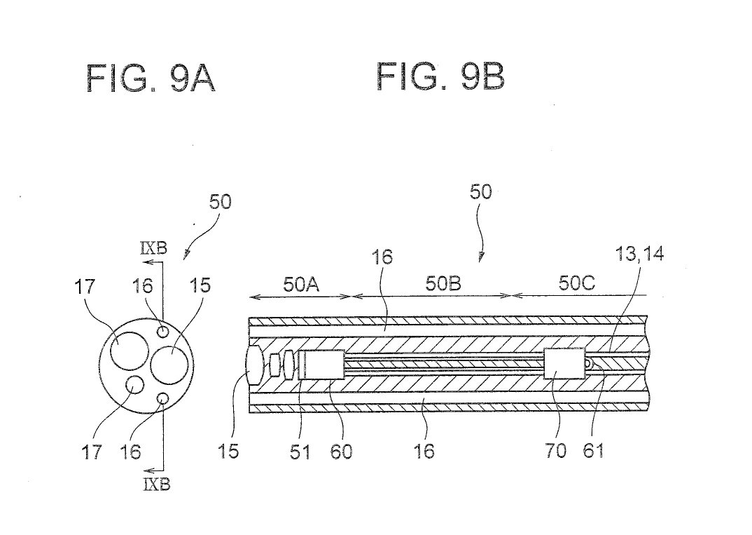

Next, the endoscope apparatus according to the second embodiment will be described below by referring to FIG. 9A and FIG. 9B. FIG. 9A is a front view showing a structure of rigid portion 50A of the scope portion 50, and FIG. 9B is a cross-sectional view taken along a line IXB-IXB in FIG. 9A, showing an internal structure of the scope portion 50. As shown in FIG. 9A and FIG. 9B, the cooling unit 60 is disposed in the rigid portion 50A and the cooling-medium circulating pump 70 is disposed in the flexible portion 50C. In this manner, by separating the cooling-medium circulating pump 70 and the cooling unit 60 to be connected to the mounting substrate 12, and by not disposing the cooling-medium circulating pump 70 of a large volume in the rigid portion 50A, it is possible to suppress an increase in a length and a diameter of the rigid portion 50A due a cooling mechanism. Furthermore, by disposing the cooling-medium circulating pump 70 not in a bending portion 50B but in the flexible portion 50C, it is possible to avoid a damage caused by an excessive stress acting on the cooling-medium circulating pump 70 or a connecting portion of the cooling-medium circulating pump 70 and the cooling-medium circulation tube 61, due to a bending operation of the bending portion 50B. Moreover, since it is also possible to make the length of the cooling-medium circulation tube 61 sufficiently substantial (long), it is possible to carry out sufficient heat release in a circulation process of the cooling medium inside the cooling-medium circulation tube 61. The cooling-medium circulating pump 70 of the second embodiment differs structurally from the cooling unit 20 used in the first embodiment, and it is possible to use a cooling medium other than an electro-conjugate fluid.

The rest of the structure, operation, and effect are similar as in the first embodiment.

### Third Embodiment

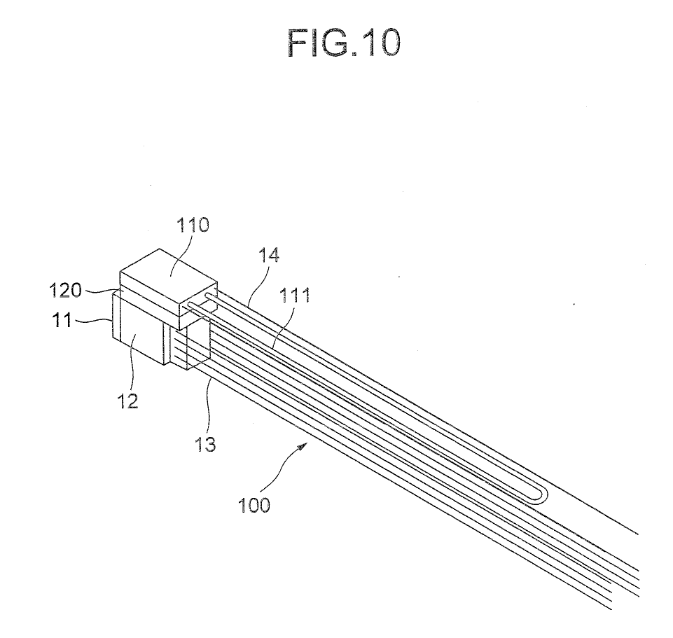

Next, an endoscope apparatus according to a third embodiment of the present invention will be described below while referring to FIG. 10. FIG. 10 is a perspective view showing an internal structure of a scope portion 100 of the endoscope apparatus according to the third embodiment, and is a diagram showing a structure of an image pickup module including the image pickup element 11. In the endoscope apparatus according to the third embodiment, a point that a Peltier element 120 is interposed between the mounting substrate 12 and a cooling unit 110 (heat absorbing section and pump) differs from the endoscope apparatus according to the first embodiment. Here, the cooling unit 110 according to the third embodiment being similar to the cooling unit 20 according to the first embodiment, the description in detail thereof is omitted. Moreover, same reference numerals are assigned to members similar as in the first embodiment, and description in detail of such members is omitted.

A heat-absorbing side of the Peltier element 120 is connected to the mounting substrate 12, and a heat-releasing side of the Peltier element 120 is connected to a bottom surface of a channel substrate of the cooling unit 110. Moreover, it is not particularly shown in the diagram but, a wire for driving is extended rearward from the Peltier element 120, and an electric power is supplied from outside of the scope portion 100.

Generally, Peltier element is useful for cooling a target member, but for using the Peltier element, it is necessary to dispose a large-size heat sink at a heat-release side. Therefore, it has been difficult to dispose it in a rigid portion of an endoscope apparatus having a limited space. However, in the endoscope apparatus according to the third embodiment of the present invention, by disposing the cooling unit 110 at the heat-release side of the Peltier element 120, an efficient heat release is possible with a compact structure which can be disposed in the rigid portion, and it is possible to cool an image pickup unit down to an environmental temperature or to a temperature less than the environmental temperature. The arrangement of a cooling-medium circulation tube 111 (circulation passage) and the cooling unit 110 in the scope 100 is similar as an arrangement of the cooling-medium circulation tube 21 and the cooling unit 20 according to the first embodiment.

As it has been described above, in the endoscope apparatus according to the third embodiment of the present invention, by combining the Peltier element 120 and an ultra-small water cooling unit including the cooling unit 20 and the cooling-medium circulation tube 111, the cooling of the image pickup unit down to the environmental temperature or a temperature less than the environmental temperature becomes possible by suppressing to the minimum, an increase in the length and the diameter of the rigid portion of the endoscope apparatus.

The rest of the structure, operation, and the effect are similar as in the first embodiment.

### Fourth Embodiment

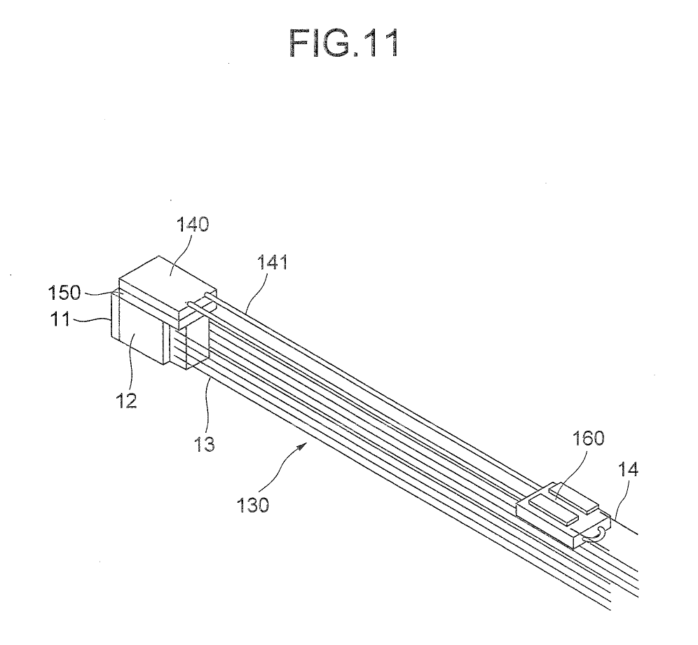

Next, a fourth embodiment of the present invention will be described below by referring to FIG. 11. FIG. 11 is a perspective view showing an internal structure of a scope portion 130 of an endoscope apparatus according to the fourth embodiment, and is a diagram showing a structure of an image pickup module including the image pickup element 11. In the endoscope apparatus according to the fourth embodiment, a point that, a Peltier element 150 (heat absorbing section) is interposed between the mounting substrate 12 and a cooling unit 140 (heat absorbing section), differs from the endoscope apparatus according to the second embodiment. Here, the cooling unit 140 according to the fourth embodiment being similar to the cooling unit 60 according to the second embodiment, the description in detail thereof is omitted. Moreover, same reference numerals are assigned to members that are similar as in the first embodiment, and the description in detail of such members is omitted.

A heat-absorbing side of the Peltier element 150 is connected to the mounting substrate 12, and a heat-releasing side of the Peltier element 150 is connected to the cooling unit 140. Moreover, it is not particularly shown in the diagram but, a wire for driving is extended rearward from the Peltier element 150, and an electric power is supplied from outside of the scope portion 130.

Even in the endoscope apparatus of the fourth embodiment, since it is possible to discharge heat efficiently from a heat releasing surface of the Peltier element 150, in a limited space inside the rigid portion similarly as in the endoscope apparatus according to the third embodiment, it is possible to cool the image pickup unit down to the environmental temperature or to a temperature less than the environmental temperature. In addition to that, a cooling-medium circulating pump 160 (pump) being disposed in a flexible portion, it is possible to suppress further an increase in the radius and the length of the rigid portion of the endoscope apparatus. An arrangement of the cooling unit 140, a cooling-medium circulation tube 141 (circulation passage), and the cooling-medium circulating pump 160 in the scope portion 130 is similar to an arrangement of the cooling unit 60, the cooling-medium circulation tube 61, and the cooling-medium circulating pump 70 according to the second embodiment.

The rest of the structure, operation, and the effect are similar as in the first embodiment and the second embodiment.

As it has been described above, in the first embodiment and the third embodiment, the cooling unit 20 has been used as a pump in which an electro-conjugate fluid is used, and for the cooling unit 110, the cooling-medium circulating pumps 70 and 160 in which a vibrator is used have been used in the second embodiment and the fourth embodiment respectively. The former is suitable for making the size small, and the latter has a merit that types of cooling media is not limited. In the present invention, these structures can be applied upon interchanging according to a size of the endoscope apparatus and a required cooling capability.

Moreover, the cooling medium filled in a circulation passage being capable of carrying out a heat exchange with the rigid portion, apart from the generation of heat by the image pickup module and the mounting substrate, even when a member which generates heat (such as an LED (light emitting diode)) is disposed in the rigid portion, it is capable of carrying out heat exchange with such member, and by releasing heat to a surrounding area of the circulation passage extended rearward from an interior of the rigid portion, it is possible to cool the rigid portion.

In this manner, since the endoscope apparatus according to the present invention is capable of cooling the rigid portion efficiently without increasing a size in a radial direction, it is useful for making the scope portion small.

Moreover, in the embodiments of the present invention, the description has been made with the image pickup module as a target of cooling. However, other heat sources such as a light guide and a light source apparatus in the tip portion of the endoscope may be let to be the target of cooling, and further, the flexible scope has been described. However, it is not restricted to the flexible scope, and may be used a rigid scope.

The endoscope apparatus according to the present invention shows an effect that it is possible to provide a cooling mechanism having a simple structure and a favorable efficiency, without the mechanism or a system becoming complicated.

What is claimed is:

1. An endoscope apparatus comprising:
a scope portion having a rigid portion in which, at least an image pickup module is disposed;
a circulation passage which is at an interior of the scope portion, and which is extended rearward from the rigid portion, such that a cooling medium filled inside is capable of carrying out a heat exchange with the rigid portion; and
a pump which is at the interior of the scope portion, and which circulates the cooling medium inside the circulation passage.

2. The endoscope apparatus according to claim 1, wherein
the scope portion includes a bending portion which is positioned rearward of the rigid portion, and which is bendable by an operation of an operator, and a flexible portion which is positioned rearward of the bending portion, and
at least a part of the circulation passage is flexible, and the circulation passage is extended at least up to the bending portion.

3. The endoscope apparatus according to claim 2, wherein the rigid portion has a mounting substrate on which, at least an image pickup element is mounted, and a part of the circulation passage is joined to the mounting substrate.

4. The endoscope apparatus according to claim 3, wherein the pump is disposed inside the rigid portion.

5. The endoscope apparatus according to claim 4, wherein the pump is joined to the mounting substrate.

6. The endoscope apparatus according to claim 2, wherein the pump is disposed inside the rigid portion.

7. The endoscope apparatus according to claim 6, wherein the pump is joined to the mounting substrate.

8. The endoscope apparatus according to claim 2, wherein the circulation passage is extended up to the flexible portion, and the pump is disposed in the flexible portion.

9. The endoscope apparatus according to claim 1, wherein the rigid portion has a mounting substrate on which, at least an image pickup element is mounted, and a part of the circulation passage is joined to the mounting substrate.

10. The endoscope apparatus according to claim 9, wherein the pump is disposed inside the rigid portion.

11. The endoscope apparatus according to claim 10, wherein the pump is joined to the mounting substrate.

12. The endoscope apparatus according to claim 1, wherein the pump is disposed inside the rigid portion.

13. The endoscope apparatus according to claim 12, wherein the pump is joined to the mounting substrate.

14. The endoscope apparatus according to claim 1, wherein the circulation passage is extended up to the flexible portion, and the pump is disposed in the flexible portion.

15. The endoscope apparatus according to claim 1, wherein a thermoelectric cooling element is disposed inside the rigid portion, and a part of the circulation passage is joined to a heat releasing surface of the thermoelectric cooling element.

16. The endoscope apparatus according to claim 1, wherein the cooling medium is an electro-conjugate fluid, and the pump sends the electro-conjugate fluid by applying an electric field to the electro-conjugate fluid.

17. An endoscope apparatus comprising:
a scope portion having a rigid portion in which, at least an image pickup module is disposed;
a heat absorbing portion which is capable of carrying out heat exchange with the rigid portion;
a circulation passage which is extended rearward from the heat absorbing portion, at an interior of the scope portion, and in which a cooling medium is filled; and
a pump which is at the interior of the scope portion, and which circulates the cooling medium in the circulation passage.
